(19) United States
(12) Patent Application Publication
(10) Pub. No.: US 2008/0280371 A1
(43) Pub. Date: Nov. 13, 2008

(54) Acoustic resonance based urea quality sensor

(75) Inventors: Ramsesh Anilkumar, Bangalore (IN); Tushar Banerjee, Bangalore (IN)

Correspondence Address: Bryan Anderson;Honeywell International Inc., 101 Columbia Rd., P.O. Box 2245, Morristown, NJ, 07962, US

(73) Assignee: Honeywell International Inc.

(21) Appl. No.: 11/803,313

(22) Filed: May 12, 2007

(51) Int. Cl. G01N 33/00; G01N 29/02

(52) U.S. Cl. 436/108; 73/19.03

(57) ABSTRACT

A urea quality sensor includes an acoustic resonator in order to measure the accurate concentration of urea by measuring change in molecular weight. A change in molecular weight of urea proportionately affects the speed of sound. The change in the composition of the urea solution manifests itself as a change in frequency. The concentration of the urea solution can be determined based on the frequency data obtained as a result of the frequency measurement utilizing the acoustic wave sensor. The urea quality sensor can be used with an NH₃ sensor in order to identify that the solution is urea.

### TECHNICAL FIELD

Embodiments are generally related to sensor methods and systems. Embodiments are also related to surface acoustic wave (SAW) devices and sensors. Embodiments are also related to sensor for determining fluid quality. Embodiments are additionally related to sense urea concentration based on acoustic resonance. BACKGROUND OF THE INVENTION

Selective Catalytic Reduction is used to inject urea—a liquid-reductant agent—through a catalyst into the exhaust stream of a diesel engine. Urea sets off a chemical reaction that converts nitrogen oxides into nitrogen and water, which is then expelled through the vehicle tailpipe. The urea quality sensor technology addresses industry quality control by ensuring that a specific quality of urea can be delivered into the exhaust gas stream. The introduction of a urea quality sensor into the selective catalytic reduction (SCR) system also reduces the risk of tampering or accidental mis-filling and helps ensure compliance, thus satisfying concerns of users and legislators alike. The urea quality sensor contributes to the overall success of SCR as a NOx reduction technology.

The urea quality sensor has been designed to monitor the quality of urea solutions used in selective catalytic reduction (SCR) systems for NOₓ emission control from diesel engines. If the engine is operated without urea solution in the onboard urea tank, excessive NOₓ emissions can occur. Using a urea quality sensor, the SCR system can be designed to prevent the possibility that the urea tank is filled with other fluids, e.g., with tap water, instead of the urea solution.

Acoustic sensors can be used to monitor the depletion of reagents and/or generation of products by measuring the speed of sound of the exhaust mixture in an acoustic cavity, which is directly related to its average molecular weight. The sensor technology exists to measure urea concentration to ensure that the fluid in the tank is urea of acceptable concentration. An NH₃ sensor could alternatively be used to ensure that urea is available in the system and is being used as needed and that the entire system is functioning properly.

It is desirable to provide an indication of urea concentration level so that the catalytic converter will perform as needed or desired. One shortcoming of previously proposed devices is that they are typically limited to very specific applications. Another limitation is that the placement of such devices is commonly limited to a supply or reservoir tank. There is a need for a more versatile arrangement that can accommodate various situations and that can be more readily incorporated into an appropriate system.

Based on the foregoing it is believed that a need exists for improved urea concentration measurement by measuring change in molecular weight using an acoustic resonance technique. By using such a methodology, measurement of urea concentration can meet customer required accuracy and resolutions.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the present invention to provide for an improved sensor.

It is another aspect of the present invention to provide for a sensor for determining fluid quality.

It is a further aspect of the present invention to provide for a method to sense liquid urea concentration based on acoustic resonance.

The aforementioned aspects and other objectives and advantages can now be achieved as described herein. A urea quality sensor includes an acoustic resonator in order to measure the accurate concentration of urea by measuring change in molecular weight. A change in molecular weight of urea proportionately affects the sound speed. The change in the composition of the urea solution manifests itself as a change in frequency. The concentration of urea solution can be determined based on the frequency data obtained as a result of the frequency measurement utilizing the acoustic wave sensor. The urea quality sensor can be used with NH₃ sensor in order to identify the solution is urea.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates an urea quality sensor with acoustic resonator, which can be utilized for the measurement of urea concentration in accordance with a preferred embodiment;

FIG. 2 illustrates a detailed view of an acoustic resonator, which can be utilized for the measurement of urea concentration in accordance with a preferred embodiment;

FIG. 3 illustrates a flowchart of operations depicting logical operational steps for sensing the concentration of urea, in accordance with an alternative embodiment.

### DETAILED DESCRIPTION

The particular values and configurations discussed in these non-limiting examples can be varied and are cited merely to illustrate at least one embodiment and are not intended to limit the scope thereof.

An acoustic resonator is a device consisting of a combination of elements having mass and compliance whose acoustic reactance cancels at a given frequency. Resonators are often used as a means of eliminating an undesirable frequency component in an acoustical system. In other instances, resonators are used to produce an increase in the sound pressure in an acoustic field at a particular frequency.

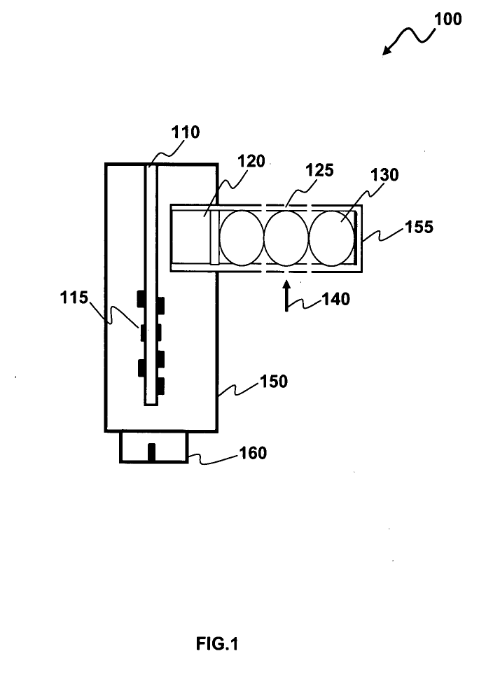

Referring to FIG. 1 a urea quality sensor 100 with acoustic resonator 120 is illustrated, which can be implemented in accordance with a preferred embodiment. The sensor 100 generally includes an acoustic resonator 120, which is generally adapted (e.g., via calibration) and used to present an acoustic standing wave 130 that can be affected by external environments (e.g., urea). The acoustic resonator can include its own housing 155 wherein environmental changes are monitored. The resonator housing 155 would logically include at least one gap 125 in the form of passages where through gases and liquids can flow. A general housing 150 can be provided to contain electronics 115 that operate in combination with the acoustic resonator 120 to make a determination regarding properties of existing urea solution 140. Electronics are mounted on a printed circuit board 110, by method known in the art. The housing 150 includes an electrical connector 160 that enables connection of the sensor 100 with other devices and/or power supplies (not shown). The connector 160 is coupled with the housing 150 and appropriate portions of the electronics on the printed circuit board 110. The housing 150 should ideally be corrosion proof, making the sensor suitable for use around corrosive liquids and gases. The sensing mechanism of the acoustic sensor 120 can contact a urea solution 140. The sensor configuration consists of an acoustic resonator 120, exhibiting a resonance frequency that is related to the velocity of sound, which, in turn, is a function of the molecular mass of urea 140.

The acoustic resonator 120 is in uniform motion at a specific frequency and amplitude. The resonator establishes an acoustic standing wave 130 (e.g., a shear wave) through its thickness. The wave pattern interacts with the urea solution 140. As the wave penetrates the surface of the urea solution 140 touching the resonator, a thin layer of fluid is set in motion absorbing power from the wave. The speed of sound in urea solution 140 can be used to measure the concentration, since the speed of sound in urea solution 140 changes with the molecular weight of the urea solution 140. The change in the molecular weight of the urea solution 140, affects sound speed proportionately. The relationship between these two quantities can be used to measure the changes of urea solution 140 concentration by measuring the speed of sound. Therefore, the speed of sound can be measured by means of the acoustic resonator 120.

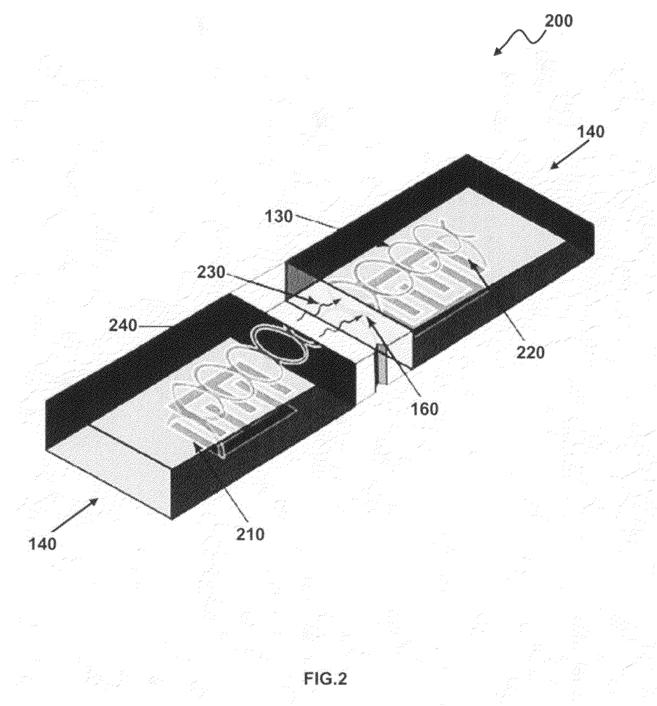

Referring to FIG. 2 a detailed view of an acoustic resonator 200 which can be utilized for the measurement of urea concentration is illustrated in accordance with a preferred embodiment. As shown in FIG. 2 interdigital transducers (IDT) 210 and 220 can be formed upon a piezoelectric substrate or layer 240. IDT 210, 220 can be configured in the form of electrodes, depending upon design considerations. A gap 260 can be formed between IDT 210 and IDT 220. In general, acoustic resonator 200 can be associated with a sensing mechanism that is communicable to urea solution 140, wherein the sensing mechanism comprises one or more acoustic wave sensing elements such as, for example, IDTs 210 and 220. One or more of the IDTs 210 and 220 can be in contact with a urea solution 140, such that the IDT associated with the urea solution 140 in responsive to an excitation of the at least one acoustic wave sensing element.

The acoustic wave resonator 200 supports a standing wave 130 through its thickness that travels from the input transducer 210 to the output transducer 220. As the vibrating surface 230 moves the characteristics of the acoustic signal changes; these changes are related to the molecular weight of urea solution 140. The output transducer 220 of the resonator 120 is in direct contact with the urea solution 140 while the input transducer 210 is hermetically sealed from the contact of urea solution 140.

The ratio of shift in frequency to original frequency can be determined as indicated by equations (1) below

$$\frac{Δ f}{f} = \frac{( {m}_{H 2 O} - {m}_{Urea} ) {X}_{H 2 O}}{2 {m}_{H 2 O}} \qquad ( 4 )$$

The resonance frequency is the frequency at which the urea solution 140 will most vigorously vibrate when driven by an external source. The speed of sound can be measured very precisely and reliably and the speed of sound of urea 140 is directly related to its chemical composition. The measurement of the speed of urea 140 can be used as a method to detect small changes in urea concentration.

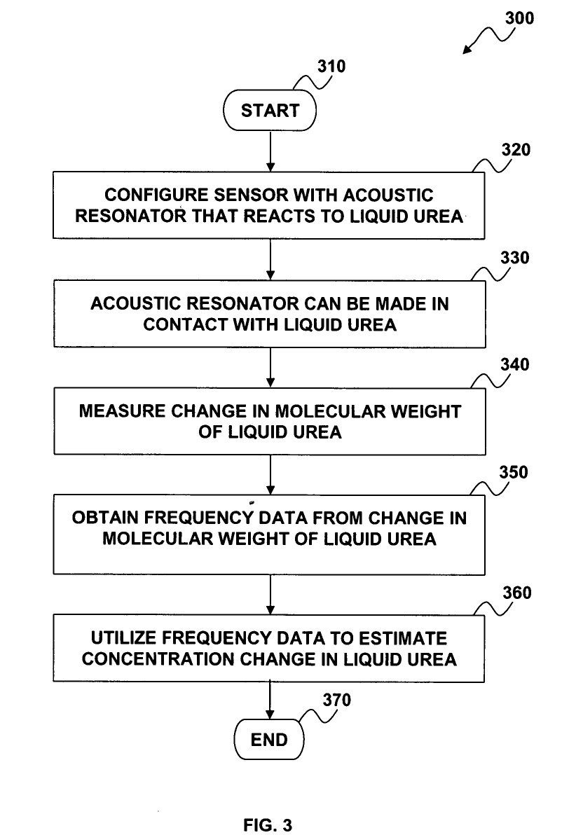

Referring to FIG. 3 a flowchart of operations depicting logical operational steps for sensing the concentration of urea 300 is illustrated, in accordance with a preferred embodiment. The process depicted in FIG. 3 can be initiated, as indicated at block 310. A sensor 100 can be configured with acoustic resonator 120 that reacts with urea solution 140, as depicted at block 320. The acoustic resonator 120 can be made in contact with urea solution 140, as illustrated at block 330. The change in molecular weight of urea solution 140 can be measured, as indicated at block 340. Thereafter, as depicted at block 350, the frequency data can be obtained from change in molecular weight. The frequency data can be utilized in order to estimate concentration change in urea solution 140, as shown at block 360. The process can then terminate, as indicated at block 370.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A method for sensing urea concentration, comprising:
providing an acoustic wave device including a first interdigital transducer and a second interdigital transducer, said acoustic wave device having a gap formed between said first inter digital transducer and said second inter digital transducer, wherein an urea solution is able to contact the gap;
Measuring change in molecular weight of said urea solution that corresponds to a change in frequency utilizing said acoustic wave device; and
Determining concentration of said urea solution based upon the molecular weight of said urea solution wherein the molecular weight and the change in frequency measurement provide data indicative of the concentration of said urea solution;

2. The method of claim 1 wherein the molecular weight and the change in frequency of said urea solution is related in accordance with the formula wherein:
$$\frac{Δ f}{f} = \frac{( {m}_{H 2 O} - {m}_{Urea} ) {X}_{H 2 O}}{2 {m}_{H 2 O}} .$$

3. The method of claim 1 wherein said acoustic wave device comprises an acoustic resonator that generates at least one bulk acoustic wave that assists in providing a measurement of said concentration of said urea solution.

4. The method of claim 3 wherein said at least one bulk acoustic wave generated by the acoustic resonator is a standing wave.

5. The method of claim 1 further comprising combining said acoustic wave sensor with a NH₃ sensor, wherein said acoustic wave device selectively reacts to said urea solution in order to provide data indicative of the presence of said urea solution.

6. A urea quality gas sensor apparatus for sensing urea concentration, comprising:
an acoustic wave device including a first interdigital transducer and a second interdigital transducer and having a gap formed therein, wherein an urea solution contacts said gap;
a housing containing said acoustic wave device wherein said housing is adapted for use in corrosive environments; and
An electrical connector extending through said housing thereby forming an electrical connection with said sensor.

7. The apparatus of claim 6 wherein said housing contains a printed circuit board with electronics coupled to the acoustic wave device.

8. The apparatus of claim 6 wherein said acoustic wave device is adapted to measure change in a molecular weight of said urea solution that corresponds to a change in frequency.

9. The apparatus of claim 6 wherein said acoustic wave device comprises an acoustic resonator that generates at least one bulk acoustic wave that assists in providing a measurement of said concentration of said urea solution.

10. The apparatus of claim 9 wherein said at least one bulk acoustic wave is a standing wave.

11. The apparatus of claim 6 wherein said acoustic wave device is adapted to determine a concentration of said urea solution based upon the molecular weight of said urea solution wherein said molecular weight and the change in frequency measurement provide data indicative of the concentration of said urea solution.

12. The apparatus of claim 6 wherein said acoustic wave device selectively reacts to said urea solution in order to provide data indicative of the presence of said urea solution.

13. A urea quality gas sensor apparatus for sensing urea concentration, comprising:
an acoustic wave device including an acoustic resonator adapted to generate at least one bulk acoustic wave to assist in providing measurement of a concentration of urea solution and a second inter digital transducer also adapted to generate at least one bulk acoustic wave to assist in providing measurement of a concentration of urea solution, said acoustic wave device having a gap formed between said acoustic resonator and said second inter digital transducer, wherein an urea solution is able to contact the gap;
A housing containing said acoustic wave device wherein said housing is adapted for use in corrosive environments; and
An electrical connector extending through said housing thereby forming an electrical connection with said sensor.

14. The apparatus of claim 13 wherein said housing contains a printed circuit board with electronics coupled to the acoustic wave device.

15. The apparatus of claim 13 wherein said acoustic wave device is adapted to measure change in a molecular weight of said urea solution that corresponds to a change in frequency.

16. The apparatus of claim 13 wherein said second interdigital transducer comprises an NH₃ sensor, wherein said acoustic wave device selectively reacts to said urea solution using said NH₃ sensor and said acoustic resonator in order to provide data indicative of the presence of said urea solution.

17. The apparatus of claim 16 wherein said at least one bulk acoustic wave generated by said acoustic resonator is a standing wave.

18. The apparatus of claim 13 wherein said acoustic wave device is adapted to determine a concentration of said urea solution based upon the molecular weight of said urea solution wherein said molecular weight and the change in frequency measurement provide data indicative of the concentration of said urea solution.

19. The apparatus of claim 18 wherein said acoustic wave device is adapted to selectively react to said urea solution in order to provide data indicative of the presence of said urea solution.

20. The apparatus of claim 13 wherein said acoustic wave device is adapted to selectively react to said urea solution in order to provide data indicative of the presence of said urea solution.
